(19) United States
(12) Patent Application Publication
(10) Pub. No.: US 2018/0125326 A1
(43) Pub. Date: May 10, 2018

(54) Domestic Appliance Having A Touch-Sensitive Operator Control Device, And Method For Operating The Same

(71) Applicant: BSH HAUSGERAETE GMBH, MUENCHEN (DE)

(72) Inventor: THOMAS NEITZEL, BERLIN (DE)

(21) Appl. No.: 15/527,534

(22) PCT Filed: Nov. 11, 2015
(86) PCT No.: PCT/EP2015/076304

§ 371 Date: May 17, 2017

(30) Foreign Application Priority Data
Nov. 17, 2014 (DE) 10 2014 223 366.6

(51) Int. Cl. A47L 15/42; A61B 5/00; D06F 39/00; F24C 7/08; F25D 29/00; G06F 3/041

(52) U.S. Cl. CPC A47L 15/4293; A61B 5/6898; A61B 5/7475; G06F 3/041; F24C 7/082; F25D 29/005; D06F 39/005

(57) ABSTRACT

A domestic appliance includes a control device and a touch-sensitive operator control device. The domestic appliance has at least one sensor with which at least one property of an external input unit can be measured. The external input unit is in contact with the operator control device or is located at a distance x from the operator control device, wherein x is lower than a prespecified maximum distance xₘₐₓ. The control device is configured to evaluate the at least one measured property with respect to the type and/or the state of the external input unit and to control the domestic appliance depending on the type and/or state of the external input unit. A method for operating the domestic appliance is also provided.

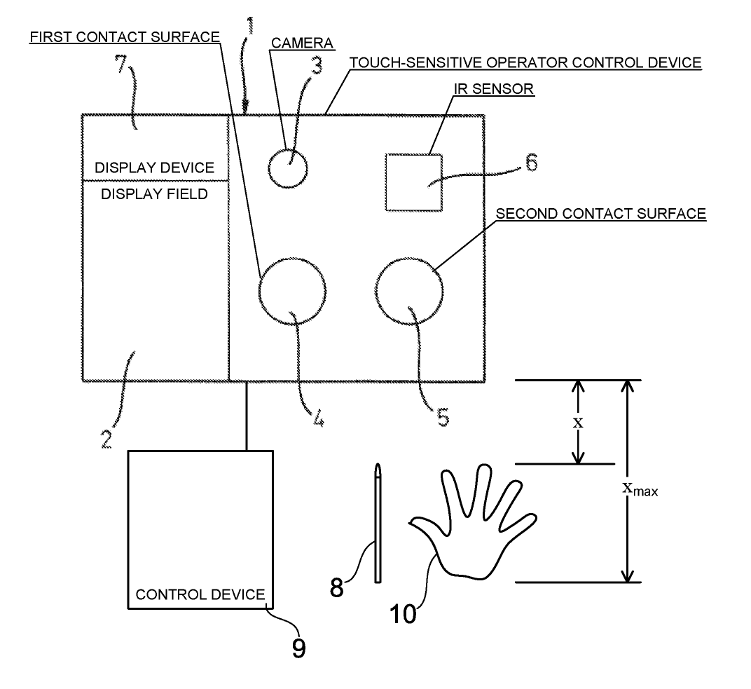

The invention relates to a domestic appliance having a touch-sensitive operator control device and a method for operating the same. The invention relates, in particular, to such a domestic appliance having a control device and a touch-sensitive operator control device as well as a preferred method for operating the same.

For the operation of computers and smartphones, it has becomes increasingly popular in recent years to use a touch-sensitive display and operator control device. Such a touch-sensitive display and operator control device which combines a display apparatus with an operator control device, and is also denoted hereinafter as a “touchscreen”, in principle permits a comfortable and rapid operation of the relevant appliance. Accordingly, already numerous patent applications and patents relate to touchscreens, for example U.S. Pat. Nos. 5,559,301, 6,130,665 and U.S. Pat. No. 6,542,171.

In the meantime, domestic appliances having a touchscreen have also become available. However, the control of a domestic appliance via a touchscreen may be very difficult when the number of programs which are able to be implemented and the number of options for setting parameters are very high. With a complex hierarchy of programs and setting options, a user of the domestic appliance may become very easily confused and lose track of the control thereof. This is the case, in particular, when many individual setting options are provided for specific program steps. However, the possibility of inputting optimal settings may permit a very efficient operation of a domestic appliance. In addition to a more efficient operation of the domestic appliance with regard to the consumption of energy and the input materials used, such as water, cleaning agent, etc. the operation may also be designed to be particularly user-friendly when it is possible to input optimal settings.

The display, i.e. the touchscreen, however, limits the range of movements which may be used for setting parameters and also limits the number of parameters which may be set by using a display. This requires a deep hierarchy of menus and windows for the purposes of setting parameters which are intended to be used for a program to be implemented in the domestic appliance. However, the control of a program is delayed thereby or methods which are not optimized are preset by a user who is perhaps not very patient, so that the domestic appliance is not operated in an optimal manner. Therefore, it is desirable to design the operation of a domestic appliance which has a touchscreen in a manner which is as efficient and as comfortable as possible.

Each user of a domestic appliance additionally has different preferences and wishes relative to the mode of operation of a domestic appliance. These preferences and wishes may refer to the design and mode of operation of a touch-sensitive operator control device, but in particular also to the operation of the domestic appliance with regard to the actual purpose of use of the domestic appliance. It would be desirable if such preferences and wishes could be taken into consideration without a great deal of assistance on the part of a user.

Moreover, even with a designated user there are emotional and health circumstances which vary over time and which have an effect on the expedient use and operation of the domestic appliance. Catering for the individual requirements of a user would improve the user-friendliness and safety of the operation of a domestic appliance. The operational life of a domestic appliance would generally be extended as a whole thereby.

The publication U.S. Pat. No. 8,398,546 B2 discloses a system for monitoring and controlling the body weight, including other physiological conditions. The system provides feedback for the purpose of weight reduction in an individual. To this end, a portable sensor for measuring data and a processing unit in electronic communication therewith are provided. Based on physiological data derived from the recorded data and other data, proposals for activities are made for an individual which are intended to permit the goal to be achieved.

The publication WO 2012/008961 A1 discloses a method and a system in order to react to stress and/or a lapse in concentration detected in a user of an electronic appliance. The method comprises the collection of information relative to the use of the electronic device by the user, the processing of information and a determination based thereon whether the user exhibits stress and/or a lapse in concentration and, in the affirmative case, the provision of multimedia content in order to counteract this.

The publication DE 10 2005 055 772 A1 discloses a personal weighing machine, a person to be weighed being able to step onto said personal weighing machine, wherein at least one hand grip is provided, the person to be weighed being able to hold onto said hand grip when stepping onto the weighing machine, wherein the hand grip comprises at least one electrical and/or optical measuring sensor. In one embodiment, an evaluation unit is provided for determining a body weight and/or a body fat content and/or a pulse frequency and/or a blood pressure of the person to be weighed.

The publication JP 2011-072644 A discloses a system and a method for detecting an abnormal state of the mind or body, for example depression.

The publication U.S. Pat. No 6,484,062 B1 discloses a computer system for combating stress and a method for the operation thereof.

With this in mind, it was the object of the present invention to provide a domestic appliance having a control device and a touch-sensitive operator control device which, even in the case of a wide selection of programs and setting options which may be implemented in the domestic appliance, may be controlled by a user himself in a simple and more rapid and more precise manner. As a result, the domestic appliance is intended to be able to be operated more efficiently as a whole. Preferably, in this case, the individual state of a user of the domestic appliance is intended to be able to be taken into consideration.

This object is achieved according to the invention by a domestic appliance and a method for the operation of the same, having the features of the corresponding independent claims. Preferred embodiments of the domestic appliance according to the invention are disclosed in the corresponding dependent claims. Preferred embodiments of the method according to the invention correspond to preferred embodiments of the domestic appliance according to the invention and vice versa, even when this is not expressly stated herein.

Therefore, the subject of the invention is a domestic appliance having a control device and a touch-sensitive operator control device, wherein the domestic appliance has at least one sensor with which at least one property of an external input unit can be measured, said external input unit being in contact with the operator control device or being located at a distance x from said operator control device, wherein x is lower than a prespecified maximum distance xₘₐₓ, wherein the control device is designed to evaluate the at least one measured property in respect of the type and/or the state of the external input unit and to control the domestic appliance depending on the type and/or state of the external input unit.

In a preferred embodiment, the external input unit is a human body, a part of a human body or an elongated input object guided by a human body part.

Preferably, the external input unit is an elongated input object, one end thereof being designed in terms of material, surface and/or shape, such that by the at least one sensor at least one property is able to be evaluated in respect of the type and/or state of the external input unit, so that the domestic appliance is able to be controlled as a function of the determined type and/or state of the external input unit. Particularly preferably, the external input unit is a stylus. The use of an elongated input object also enables a person, who is restricted regarding their options for physical expression, to control a domestic appliance, by taking into account their state, in particular their state of health and/or emotional state. For example, a set of different elongated input objects which differ with regard to their properties could be provided for the operation of a domestic appliance. For example, the elongated input objects could differ in their resistance or in their contact surface, i.e. the size of surface via which the external input unit comes into contact and/or is able to come into contact with the touch-sensitive operator control device. A different state of health or state of mind or even a stressed state or a state of a lapse in the ability to concentrate may be assigned to each of these input objects. Such an embodiment, however, requires a conscious decision on the part of a user of the domestic appliance.

The sensor according to the invention is not particularly limited as long as it is able to fulfill the object set according to the invention. In particular, the measuring principles may differ. Moreover, the sensor may be an individual sensor or a sensor system comprising a plurality of sensors. Finally, such a sensor system may also be a radiation unit for radiation, in particular IR radiation, as well as a receiving unit for the radiation reflected by the external input unit, in particular reflected IR radiation.

In a preferred embodiment of the domestic appliance, at least one sensor is a camera which is attached to the domestic appliance and the at least one property is a gesture or facial expression of a human body located at the distance x from the camera, wherein 0<x<xₘₐₓ. In this case, the camera is preferably arranged in a plane which is substantially equal to the operator control device or behind the operator control device in the domestic appliance. Such an embodiment also generally requires a conscious decision on the part of a user of the domestic appliance. The term “gesture” is to be interpreted broadly here. For example, it may be a specific position of a hand or both hands of a user or a body position, for example an upright position to the side of the camera or with the back and/or the front of the user toward the camera. The facial expression may be characterized, for example, by a smile, a laugh, a sad state or a grimace. In this embodiment, software, which is able to identify and evaluate the gestures or facial expressions present, is generally stored in the control device. In this case, it may also be preferably provided that the user of an external input unit is informed that although the presence of an external input unit is identified, the use thereof by the user does not permit any measurement and/or evaluation and therefore results in a changed use.

According to the invention, it is further preferred if the properties of an input unit of the domestic appliance which is not able to be consciously controlled by a user may be measured and evaluated for the control.

In a further preferred embodiment, the external input unit is a human hand or a part thereof and the at least one property encompasses a size of a contact surface on the touch-sensitive operator control device and/or one or more biophysical properties of the human body or a part thereof. In this case the one or more biophysical properties are preferably selected from the group which consists of body temperature, pulse, electrocardiogram, blood pressure, moisture, glucose content in the blood, oxygen content in the body or in the blood, fat content and electrical resistance. In this case the term “moisture” in particular relates to sweat, primarily hand sweat. This may be a measurement of the state of health or stress of a user.

In one embodiment of the invention, the measured biophysical properties may also be used for identifying a user. In this embodiment, advantageously specific information for this user may be stored in the control device of the domestic appliance, for example numerical values of the biophysical properties which may be assigned to different health and/or emotional states of the user. Also, the preferences or wishes of the user for the mode of operation of the domestic appliance may also be stored.

The measurement of biophysical properties is known per se to the person skilled in the art. Depending on the property to be measured, sensors which react to contact, i.e. touch-sensitive sensors, may be used for the measurement, wherein if required not only the finger of one hand is sufficient for the contact but also a finger of the second hand has to be used for the measurement. Then a corresponding suggestion is given to the user, preferably by means of a display device of the domestic appliance. Thus for detecting the pulse rate of a user the voltage produced by the heart of the user between the two hands may be detected. This may be carried out, for example, by two fingers of two hands being placed on contact surfaces of the touch-sensitive operator control device provided therefor.

However, sensors which emit electromagnetic radiation and measure reflected radiation may also be used. For example, an IR sensor which comprises an IR radiator and an IR receiving device could be used. If namely the sensor has a light source, such as for example an LED (light emitting diode) or a laser, in particular a semiconductor laser, for example, this may be used for measuring the pulse. Thus, for example, the pulse rate of the user and thus the pulse frequency may also be optically determined. This may be carried out, for example, by the back-scattering characteristic, which changes due to the pulse rate, and/or the reflectivity of the human tissue, which changes over time due to the pulse rate, being measured and evaluated.

Moreover, with a combination of different sensors, for example, it is possible to measure the blood pressure of the user via the temporal shift of an electrically measured voltage signal of the pulse rate and an optically measured signal of the pulse rate.

Finally, an IR sensor (infrared sensor) may also be used for measuring the body temperature of a user. In such an embodiment, for example, an infrared sensor which consists of an infrared source, i.e. an IR radiator, and an infrared receiving appliance, i.e. an IR receiving device, is present in the touch-sensitive operator control device.

Moreover, with the presence of such an optical sensor a photoplethysmogram may also be recorded in order to measure and to examine the vein system of a person and, for example, to measure the blood pressure. The photoplethysmogram, also denoted as light reflection rheography, is a non-invasive screening method for assessing the function of the vein system in the extremities of a person, for example a finger. In this case infrared light is radiated into the skin at a defined wavelength, with maximum absorption in the near infrared range, and subsequently the reflected component is registered and plotted. The proportion of reflected infrared light is dependent on the filling state of the cutaneous venous plexus which changes during a movement program. Such a non-invasive measurement of the blood pressure is disclosed, for example, in DE 10043266 A1.

In a particularly preferred embodiment, the domestic appliance contains an optical and/or acoustic display device. In this case, said display device is preferably designed to inform a user to what extent the at least one property of the external input unit may be measured and evaluated. Thus, for example, a domestic appliance could possibly detect the presence of an external input unit but possibly not measure the properties thereof, since in embodiments of the invention, for example, the extent of the contact with the touch-sensitive operator control device is insufficient or a gesture is too unclear. It could also be provided that the display unit emits a recommendation to the user of the domestic appliance of how the external input unit is to be used.

The touch-sensitive operator control device in the domestic appliance according to the invention is not limited. Thus a touch-sensitive operator control device which is based on a change in the resistance, a change in the pressure or a change in the capacitance may be used. According to the invention, preferably the use of a capacitive operator control device is preferred. The touch-sensitive operator control device has at least one touch-sensitive surface, a control of the domestic appliance being implemented thereby. The size of this surface, which is also denoted herein as the contact surface, is generally selected such that contact by a finger, a hand or an input object may be easily detected. In this case, the sensors provided according to the invention may be integrated in such touch-sensitive surfaces, i.e. contact surfaces, or may be present as contact surfaces which are independent thereof. Depending on the biophysical property to be measured, one or more contact surfaces may be provided for measuring a biophysical property.

In a preferred embodiment of the domestic appliance, the type and/or state of the external input unit encompasses a state of health, a lapse in concentration, a stressed state and/or a negative or positive state of mind. A negative state of mind might in this case, for example, be a depressed mood or a state of increased irritability. In this case, such states may also be combined into groups, enabling improved and straightforward control of the domestic appliance. “Straightforward control” means, for example, that a specific measure to be carried out in the domestic appliance in the control unit is assigned to a group of states.

Preferably, the control unit is designed to control the domestic appliance such ii that an operating program is carried out in the domestic appliance and/or the operability of the touch-sensitive operator control device is implemented with regard to the menu selection and design as a function of the type and/or state of the external input unit. The operating program and the design of the touch-sensitive operator control device in this case are dependent on the domestic appliance. In any case, the complexity or simplicity of the selection of programs to be implemented in the domestic appliance and the size, color, brightness value and/or contrast value of a selected display are included in the design of the touch-sensitive operator control device.

The domestic appliance according to the invention is not particularly limited. Particularly preferably, however, the domestic appliance has numerous possibilities for a program to be implemented therein, so that for a user of the domestic appliance the use thereof may be complicated. The present invention then proves particularly advantageous.

An example of a domestic appliance according to the invention is a domestic appliance in which food is prepared, for example an oven, or a domestic appliance in which food is stored, for example a refrigerator.

The domestic appliance according to the invention may, in particular, also be a water-conducting domestic appliance, having a container for receiving objects to be treated, which is selected from the group which consists of a washing machine, a washer-dryer, a dryer and a dishwasher.

Depending on the type thereof, the domestic appliance according to the invention generally has at least one further sensor for determining a significant characteristic for the operation of the domestic appliance. If the domestic appliance is a washing machine, for example, suitable sensors could determine the type and quantity of laundry load and the degree of soiling thereof. In a washer-dryer, for example, in addition to the type and quantity of laundry load, the sensors could also determine the level of humidity of the laundry. Using such data, the control device could evaluate specific methods and enable this evaluation to influence the operation of the domestic appliance by means of the touch-sensitive operator control device. For example, the control device could be designed such that the selection of program settings, which are regarded as negative by the control device for the domestic appliance, is impeded or negative program settings are characterized as such for a user.

In a preferred embodiment according to the invention, the domestic appliance is designed to implement a program, i.e. an operating program, with improved hygiene action as a priority, when detecting a diseased state of a user. The operating program in this case is dependent on the type of domestic appliance. A program with a hygiene action is, in particular, implemented in a washing machine, a washer-dryer or a dryer. An improvement of the hygiene action may be achieved, in particular, by setting a higher temperature, optionally for a longer time period. Other measures may also be provided, such as the addition of a disinfecting substance or irradiation by disinfecting radiation, for example UV light.

If the domestic appliance is a refrigerator, when detecting a diseased state and/or obesity as a result of the measurement and evaluation provided according to the invention a user could be provided with a dietary recommendation, wherein it is preferably stored in a memory device of the refrigerator which foods and drinks are kept therein.

In a preferred embodiment of the domestic appliance, in the case of a negative state of mind, a stressed state or a lapse in concentration, the operator control device displays a simplified menu selection and/or the menu selection is designed to be colorful, with modified sizes of symbols or characters, with a modified brightness value and/or a modified contrast ratio. Thus, for example, in the case of a negative state of mind the menu selection could be displayed by using warm and cheerful colors. Accordingly, “blue” is designed to have a calming effect. Also reducing the rotational speed in a washing machine or a laundry dryer could provide a quieter operation of the domestic appliance, for example. Finally, an acoustic display device could also provide pleasant background noise, for example with relaxing or stimulating/exciting music.

If the identity of the user has also been determined by measuring the information of the external input unit, when storing favorite colors and/or favorite music of the determined user, the menu selection and the display device could be operated so as to be adapted thereto.

In a further preferred embodiment of the domestic appliance and, in particular, also a method according to the invention described below, with reference to the control device it is determined whether a measured property is within a permitted range. When detecting that this measured property is outside a permitted range, a further operation of the domestic appliance might not be possible and/or the control device could be designed to trigger an alarm. In this case, the term “alarm” is to be broadly interpreted. Here it may be an optical and/or acoustic alarm signal of an acoustic and/or optical display device. Alternatively or in addition, the domestic appliance could transmit a warning signal, for example via a telephone line or the Internet.

In a preferred embodiment of the invention, therefore, the control device is designed to determine whether a measured property is within a permitted range and, when detecting that this measured property is outside a permitted range, to prevent further operation of the domestic appliance and/or to trigger an alarm.

In a further preferred embodiment, the control device is designed to evaluate the contact with regard to identifying a user and the stored preferences thereof. This may be implemented, for example, by the recognition of a fingerprint being provided in the touch-sensitive center. After the assignment of the fingerprint to a person, the further menu design may then be stored on the touchscreen, i.e. the sequence of program clusters, subclusters, etc. by taking into account preferences stored in the control device.

In embodiments of the invention, the domestic appliance, generally a control device inserted therein, may identify the preferred decisions of a user and, with subsequent use of the domestic appliance, use them in order to optimize the decision situations. This may be implemented in the touch-sensitive control device, for example, by specific menu proposals being shown or preferred programs and options being highlighted.

A further subject of the invention is a method for operating a domestic appliance having a control device and a touch-sensitive operator control device, wherein the domestic appliance has at least one sensor with which at least one property of an external input unit can be measured, said external input unit being in contact with the operator control device or being located at a distance x from said operator control device, wherein x is lower than a prespecified maximum distance _{xmax,} and wherein the control device is designed to evaluate the at least one measured property in respect of the type and/or the state of the external input unit and to control the domestic appliance depending on the type and/or state of the external input unit, wherein at least one property of an external input unit is measured by the at least one sensor, said external input unit being in contact with the operator control device or being located at a distance x from said operator control device, wherein x is lower than a prespecified maximum distance xₘₐₓ, and the control device evaluates the at least one measured property in respect of the type and/or the state of the external input unit and controls the domestic appliance depending on the type and/or state of the external input unit.

The advantage of the invention is that a domestic appliance may be controlled in a simple and straightforward manner. By taking into account the physical and/or mental situation of a user and, in embodiments, the identity thereof, an adapted selection of operating programs may be displayed and/or a specific operating program may be automatically implemented. In embodiments of the invention in which the identity of the user is determined, with the availability of stored properties and preferences of the user, a further improved adaptation of the mode of operation of the domestic appliance may be implemented.

As a result, it is possible to operate a domestic appliance with improved safety but also with improved handlability (user-friendliness).

This also provides, in particular, a stronger emotional link on the part of a user to the domestic appliance. Generally, the acceptability of new domestic appliances or operating concepts may be improved.

The invention is illustrated hereinafter with reference to a touch-sensitive operator control device, shown in the only figure, of a non-limiting embodiment of a domestic appliance according to the invention.

The figure shows a touch-sensitive operator control device 1 having a display field 2 with a program menu. In the display field 2, touch-sensitive surfaces, i.e. contact surfaces, may be arranged spatially in front of the displays of operating programs which are able to be implemented in the domestic appliance and options therefor. Otherwise, no further details are provided for the design of the display field. An acoustic and/or optical display device 7 is also located above the display field 2. In this case, in addition to the possibility of emitting acoustic signals, in contrast to the display field 2, a user may also be informed about an operating state of the domestic appliance or, for example, a request may be made with regard to the correct use of an external input unit. No touch-sensitive contact surfaces are located in the display device 7 shown here.

Four sensors 3 to 6 are arranged in the touch-sensitive operator control device 1 of the figure, with which sensors at least one property of an external input unit can be measured, said external input unit being in contact with the operator control device 1 or being located at a distance x from said operator control device, wherein x is lower than a prespecified maximum distance xₘₐₓ. 3 in this case is a camera for measuring a gesture or a facial expression of a user of the domestic appliance. 4 is an IR sensor, which in this case comprises an IR radiator and an IR receiving device, not shown further. The IR sensor 4 may, as described above, be used for measuring a body temperature or a blood pressure, for example. 4 denotes a first contact surface for an external input unit and contains a first touch-sensitive sensor. 5 denotes a second contact surface for an external input unit and contains a second touch-sensitive sensor. If biophysical properties are to be measured, different biophysical properties may be assigned to the two sensors and/or contact surfaces 5 and 6. Individual biophysical properties may, however, also require that body parts which are located as far apart as possible from one another, for example a finger of the left hand and/or a finger of the right hand, come into contact with the two sensors and/or contact surfaces 5 and 6. Optionally, to this end a request may be issued by the display device 7 to a user.

### LIST OF REFERENCE NUMERALS

1 Touch-sensitive operator control device, for example capacitive operator control device
2 Display field with program menu
3 Camera
4 First contact surface for an external input unit; first (touch-sensitive) sensor
5 Second contact surface for an external input unit; first (touch-sensitive) sensor
6 IR sensor (comprising an IR radiator and an IR receiving device)
7 Acoustic and/or optical display device

1-15. (canceled)

16. A domestic appliance, comprising:
a touch-sensitive operator control device;
at least one sensor for measuring at least one property of an external input unit being in contact with said operator control device or being located at a distance x from said operator control device, the distance x being less than a prespecified maximum distance xₘₐₓ; and
a control device being configured to evaluate said at least one measured property with respect to at least one of a type or state of the external input unit, said control device being configured to control the domestic appliance depending on at least one of the type or the state of the external input unit.

17. The domestic appliance according to claim 16, wherein the external input unit is a human body, a part of a human body or an elongated input object guided by a human body part.

18. The domestic appliance according to claim 16, wherein the external input unit is an elongated input object having an end, the end having at least one of a material, a surface or a shape permitting said at least one sensor to evaluate said at least one property with respect to at least one of the type or the state of the external input unit for controlling the domestic appliance as a function of at least one of a determined type or state of the external input unit.

19. The domestic appliance according to claim 18, wherein the external input unit is a stylus.

20. The domestic appliance according to claim 17, wherein said at least one sensor is a camera which is attached to the domestic appliance, and said at least one property is a gesture or a facial expression of a human body located at the distance x from said camera, wherein 0<x<xₘₐₓ.

21. The domestic appliance according to claim 17, wherein the external input unit is a human hand or a part of a human hand, and said at least one property encompasses at least one of a size of a contact surface on said touch-sensitive operator control device or one or more biophysical properties of a human body or a part of a human body.

22. The domestic appliance according to claim 21, wherein said one or more biophysical properties are selected from the group consisting of body temperature, pulse, electrocardiogram, blood pressure, moisture, glucose content in blood, oxygen content in the body or in the blood, fat content and electrical resistance.

23. The domestic appliance according to claim 16, which further comprises at least one of an optical display device or acoustic display device.

24. The domestic appliance according to claim 23, wherein said display device is configured to inform a user to what extent said at least one property of the external input unit may be measured and evaluated.

25. The domestic appliance according to claim 16, wherein the at least one of the type or state of the external input unit encompasses at least one of a state of health, a lapse in concentration, a stressed state or a negative or positive state of mind.

26. The domestic appliance according to claim 16, wherein said control device is configured to control the domestic appliance by at least one of carrying out an operating program in the domestic appliance or implementing an operability of said touch-sensitive operator control device with regard to a menu selection and configuration as a function of the at least one of the type or state of the external input unit.

27. The domestic appliance according to claim 26, wherein the domestic appliance is configured to implement a program with improved hygiene action as a priority, when detecting a diseased state of a user.

28. The domestic appliance according to claim 26, wherein said operator control device displays at least one of a simplified menu selection, a colorful menu selection, modified sizes of symbols or characters, a modified brightness value or a modified contrast ratio, upon encountering a negative mental state, a stressed state or a lapse in concentration of the user.

29. The domestic appliance according to claim 16, wherein said control device is configured to determine if said at least one measured property is within a permitted range and to at least one of prevent further operation of the domestic appliance or to trigger an alarm when detecting that said at least one measured property is outside said permitted range.

30. A method for operating a domestic appliance, the method comprising the following steps:
providing a touch-sensitive operator control device of the domestic appliance;
placing an external input unit in contact with the operator control device or at a distance x from the operator control device, the distance x being less than a prespecified maximum distance xₘₐₓ;
using at least one sensor of the domestic appliance to measure at least one property of the external input unit; and
using a control device of the domestic appliance to evaluate the at least one measured property with respect to at least one of a type or state of the external input unit and using the control device to control the domestic appliance depending on at least one of the type or the state of the external input unit.
